(19) United States
(12) Patent Application Publication
(10) Pub. No.: US 2003/0187433 A1
(43) Pub. Date: Oct. 2, 2003

(54) Rotary device for fixing vertebrae under treatment

(75) Inventor: Chih-I Lin, Chino Hills, CA (US)

Correspondence Address: BACON & THOMAS, 4th Floor, 625 Slaters Lane, Alexandria, VA, 22314, US

(73) Assignee: A-Spine Inc., Tokyo (JP)

(21) Appl. No.: 10/107,399

(22) Filed: Mar. 28, 2002

(51) Int. Cl. A61B 17/58

(52) U.S. Cl. 606/61

(57) ABSTRACT

A rotary device includes a fastening seat and a fastening block. The fastening seat is provided with a receiving slot for receiving a spine-locking rod or screw. The receiving slot is provided on the inner wall with two retaining recesses. The fastening block is provided with a retaining flange and an arcuate press surface. The fastening block is disposed in the receiving slot of the fastening seat. As the fastening block is so turned that the retaining flange of the fastening block is fitted into the retaining recesses of the fastening seat, the spine-locking rod or screw is pressed against by the arcuate press surface of the fastening block.

### FIELD OF THE INVENTION

The present invention relates generally to a spinal fixation device, and more particularly to a device which can be rotated to fix a vertebral fixation rod or screw.

### BACKGROUND OF THE INVENTION

The vertebral surgery is often carried out in conjunction with one or more locking rods, such as those which are disclosed by this inventor of the present invention in the U.S. Pat. Nos. 5,387,212 and 5,582,612. The locking rods can be used in conjunction with other fixation members, as disclosed in the U.S. Pat. Nos. 5,380,362 and 5,476,462.

As shown in FIG. 11, the above U.S. Pat. No. 5,380,326 discloses a structure comprising a U-shaped outer clamping member 80 and an inner clamping member 85. The outer clamping member 80 is provided in the interior with a slightly-inclined recess 82, whereas the inner clamping member 85 is provided in the exterior with a slightly-inclined projection 86, which is fitted into the recess 82 of the outer clamping member 80 at the time when the inner clamping member 85 is slid into the outer clamping member 80, so as to fix a locking rod which is disposed in the U-shaped opening of the outer clamping member 80.

As shown in FIG. 12, the afore-mentioned U.S. Pat. No. 5,476,462 discloses a structure comprising an outer clamping member 90 and an inner clamping member 95. The outer clamping member 90 is provided in the interior with a slightly-inclined slot, whereas the inner clamping member 95 is provided in the exterior with a slightly-inclined surface. A locking rod is securely held by the structure at the time when the outer clamping member 90 and the inner clamping member 95 are joined together such that the inclined surface of the inner clamping member 95 is fitted into the inclined slot of the outer clamping member 90.

These two prior art clamping structures described above are defective in design in that they can not be easily maneuvered by a surgeon in operation, and that the locking rod is susceptible to displacement at such time when the inner clamping member is finally pushed to locate securely in the interior of the outer clamping member. It is conceivable that the displacement of the locking rod has an adverse effect on the outcome of the surgery.

### SUMMARY OF THE INVENTION

The primary objective of the present invention is to provide a vertebral fixation device which is free of the deficiencies of the prior art devices described above.

In keeping with the principle of the present invention, the foregoing objective of the present invention is attained by a rotary fixation device comprising:

a fastening seat provided with a U-shaped slot for receiving the spine-locking rod or screw, said U-shaped slot provided on two opposite inner walls with an inner retaining recess; and

a fastening block provided with a retaining flange which is provided in two opposite sides with a tangent surface, said fastening block further provided in a bottom with an arcuate press surface, and in a top with a rotating portion, wherein said fastening block is rotatably joined with said fastening seat such that said fastening block can be turned so as to enable said retaining flange to fit into said inner retaining recesses of said fastening seat, thereby causing said arcuate press surface of said fastening block to press against the spine-locking rod or screw which is received in said U-shaped slot of said fastening seat.

Preferably, said U-shaped slot of said fastening seat is provided on two opposite outer walls with an outer retaining recess to facilitate the holding of said fastening seat by a hand tool.

Preferably, said rotating portion is provided with a position-confining groove; wherein said U-shaped slot of said fastening seat is provided at a top of two side walls thereof with a position-confining protrusion, wherein said position-confining protrusion is fitted into said position-confining groove of said rotating portion of said fastening block at such time when said fastening block is mounted to said U-shaped slot of said fastening seat.

Preferably, said arcuate surface of said fastening block is provided with an assembly arcuate recess parallel to said tangent surface, and a fixation arcuate recess perpendicular to said assembly arcuate recess, with said assembly arcuate recess being greater in depth than said fixation arcuate recess. More preferably, said arcuate press surface of said fastening block is further provided with an adjustment arcuate recess whereby said adjustment arcuate recess is located between said assembly arcuate recess and said fixation arcuate recess and is intermediate in depth between said assembly arcuate recess and said fixation arcuate recess.

Preferably, said fastening seat is provided with a fastening screw made integrally therewith.

Preferably, said fastening seat is provided with a clamp made integrally therewith.

Preferably, said fastening seat is provided with a receiving hole below the U-shaped slot, and is further provided with a connection apparatus comprises a spherical head and a press ring, wherein said spherical head and said press ring are adapted to be received in said receiving hole with said press ring resting on said spherical head, and said spherical head and said press ring will be pressed securely in the fastening seat by the spine-locking rod or screw, when the fastening block is joined with said fastening seat. More preferably, said receiving hole is a deviation hole, and said fastening seat is further provided with an assembly ring, wherein said assembly ring which is adapted to mounted to said receiving hole so that said spherical head is clamped between said assembly ring and said press ring. Preferably, said spherical head is provided with a fastening screw made integrally therewith, wherein said fastening screw is extruding from said receiving hole when said spherical head is received in said receiving hole.

Preferably, said fastening block is provided with a threaded hole for threadably receiving a fastening screw for resulting an added pressure to exert on the spine-locking rod or screw.

The features and the advantages of the present invention will be more readily understood upon a thoughtful deliberation of the following detailed description of the preferred embodiments of the present invention with reference to the accompanying drawings.

### BRIEF DESCRIPTION OF THE DRAWINGS

FIG. 1 shows an exploded view of a first preferred embodiment of the present invention.

FIG. 2 shows a structural schematic view of the bottom of the fastening block of the first preferred embodiment of the present invention.

FIG. 3 shows a perspective view of the first preferred embodiment of the present invention in combination.

FIG. 4 shows a sectional view of a portion taken along the direction indicated by a line 4-4 as shown in FIG. 3.

FIG. 5 shows an exploded view of a second preferred embodiment of the present invention.

FIG. 6 shows a perspective view of the second preferred embodiment of the present invention in combination.

FIG. 7 shows an exploded view of the first preferred embodiment of the present invention in conjunction with a pivoting device.

FIG. 8 shows a longitudinal sectional view of the first preferred embodiment of FIG. 7 at work.

FIG. 9 shows a longitudinal sectional view of the first preferred embodiment of the present invention at work in conjunction with another pivoting device.

FIG. 10 shows an exploded view of a third preferred embodiment of the present invention.

FIG. 11 shows an exploded view of the structure of the U.S. Pat. No. 5,380,326.

FIG. 12 shows a perspective view of the structure of the U.S. Pat. No. 5,476,462.

### DETAILED DESCRIPTION OF THE PREFERRED EMBODIMENTS

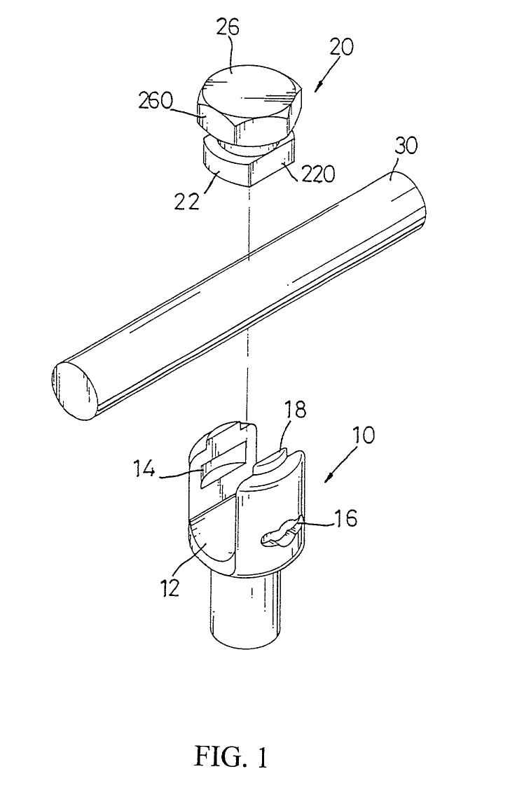

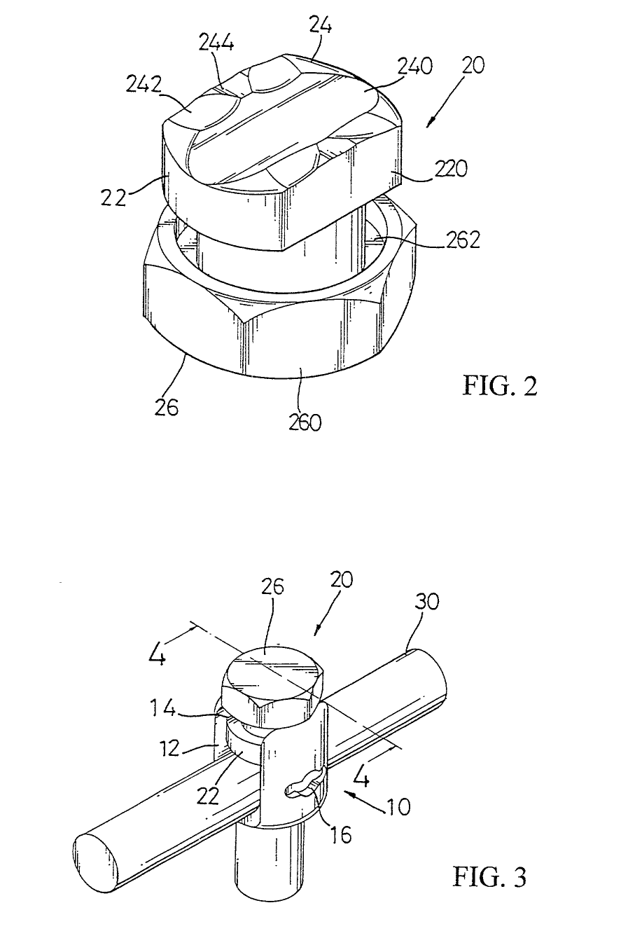

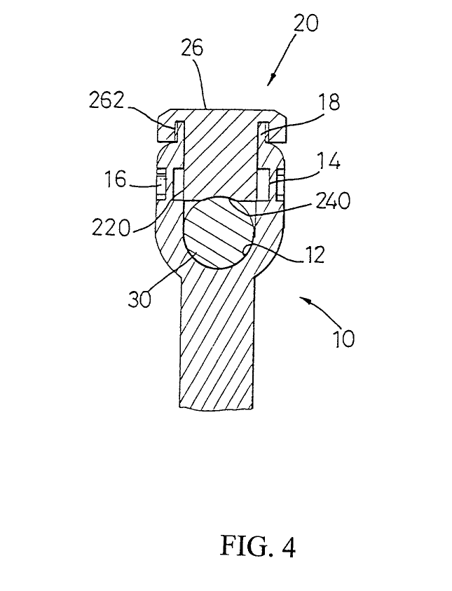

As shown in FIGS. 1-4, a rotary device of the first preferred embodiment of the present invention comprises a fastening seat 10 and a fastening block 20.

The fastening seat 10 has a U-shaped slot 12 in which a vertebra-locking rod 30 is received. The slot 12 is provided on two opposite inner walls with an inner retaining recess 14, and on two opposite outer walls with an outer retaining recess 16. The U-shaped slot 12 is provided at the top of each of two side walls thereof with a position-confining protrusion 18.

The fastening block 20 is provided in the periphery of the bottom portion thereof with a retaining flange 22 which is provided in two opposite sides with a tangent surface 220. The fastening block 20 is provided on the underside with an arcuate press surface 24, and in the upperside with a rotating portion 26. The arcuate press surface 24 is provided with an assembly arcuate recess 240 extending along the direction of the tangent surface 220, and a fixation arcuate recess 244 perpendicular to the assembly arcuate recess 240. Located between the assembly arcuate recess 240 and the fixation arcuate recess 244 is an adjustment arcuate recess 242. The assembly arcuate recess 240 has the greatest depth. The fixation arcuate recess 244 has the smallest depth. The adjustment arcuate recess 242 has a depth which is intermediate between the depth of the assembly arcuate recess 240 and the depth of the fixation arcuate recess 244.

The rotating portion 26 of the fastening block 20 has a hexagonal profile 260. The fastening block 20 is further provided with a position-confining groove 262.

In operation, a vertebra-locking rod 30 is received in the U-shaped slot 12 of the fastening seat 10. Thereafter, the fastening block 20 is disposed in the U-shaped slot 12 of the fastening seat 10 such that the locking rod 30 is pressed against by the assembly arcuate recess 240 of the arcuate press surface 24 of the fastening block 20, and that the retaining flange 22 of the fastening block 20 is adjacent to the inner retaining recesses 14 of the fastening seat 10. As the fastening block 20 is turned by a hand tool which holds the rotating portion 26 of the fastening block 20, the retaining flange 22 of the fastening block 20 is fitted into the inner retaining recesses 14 of the fastening seat 10, thereby causing the arcuate press surface 24 to press against the locking rod 30. When the fastening block 20 is so turned that the locking rod 30 is rested on by the adjustment arcuate recess 242, the locking rod 30 is pressed against fairly well. However, the locking rod 30 has not yet been fixed completely. As a result, the locking rod 30 still can be properly adjusted and located before the fastening block 20 is turned once again such that the locking rod 30 is pressed against by the fixation arcuate recess 244, and that the locking rod 30 is firmly held between the U-shaped slot 12 of the fastening seat 10 and the fixation arcuate recess 244 of the fastening block 20. In addition, when the fastening block 20 is mounted to the U-shaped slot 12 of the fastening seat 10, the position-confining protrusion 18 of the fastening seat 10 is fitted into the position-confining groove 262 of the rotating portion 26 of the fastening block 20, thereby resulting in an added fixation effect.

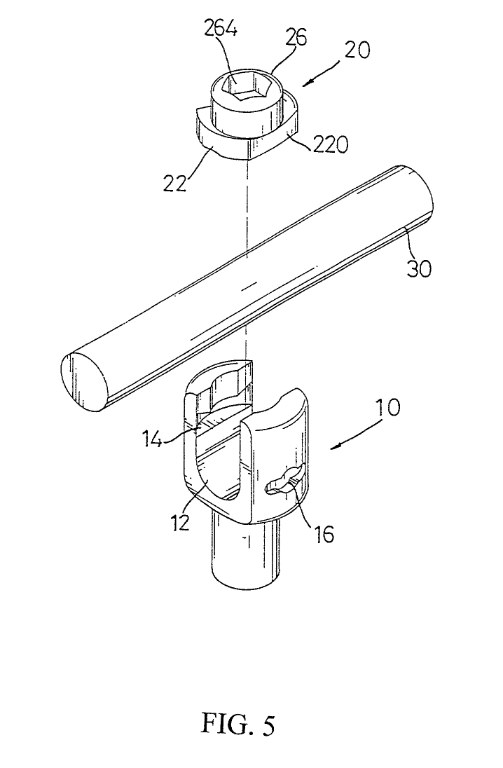

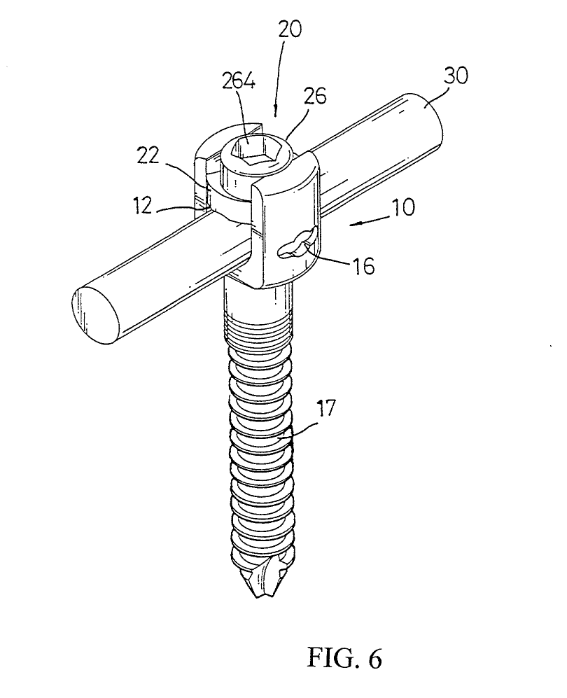

As shown in FIGS. 5 and 6, a rotary device of the second preferred embodiment of the present invention is basically similar in construction to that of the first preferred embodiment as described above, except that the former comprises a fastening seat 10 which is devoid of the position-confining protrusion 18, and a fastening block 20 which is provided in the rotating portion 26 with a hexagonal recess 264 and is devoid of the position-confining groove 262. In addition, the fastening seat 10 of the second preferred embodiment is provided with a fastening screw 17 which is made integrally therewith and is cooperative with the device disclosed in the U.S. Pat. No. 5,439,463.

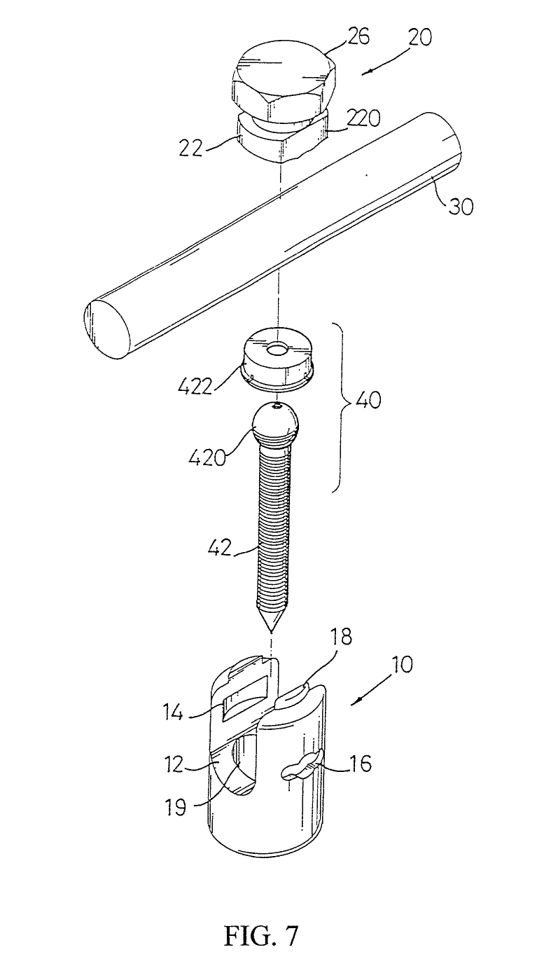

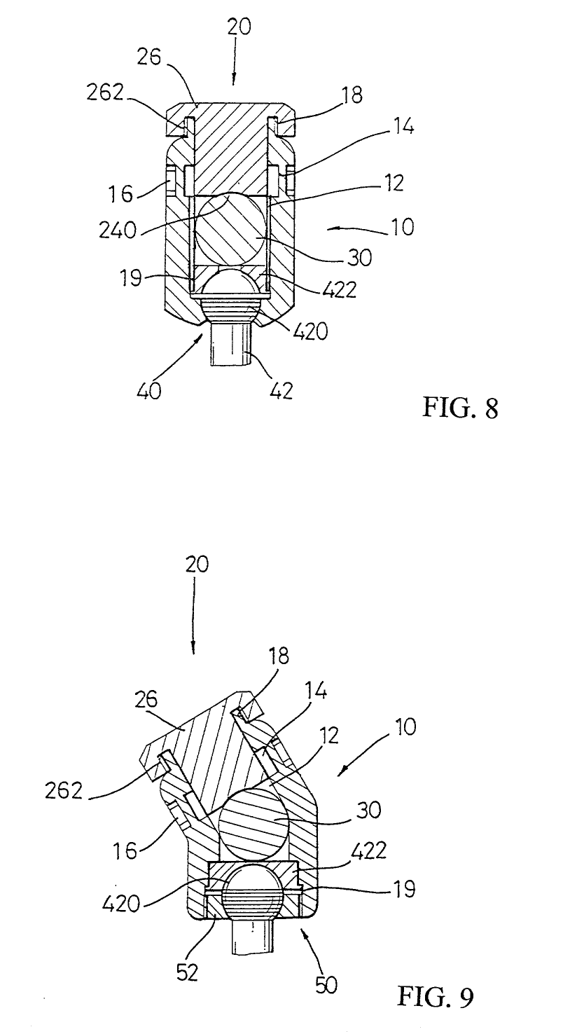

As shown in FIGS. 7 and 8, the device of the first preferred embodiment of the present invention is used in conjunction with a connection apparatus 40 comprising a fixation screw 42 with a spherical head 420, and a press ring 422. The fastening seat 10 is provided with a receiving hole 19 for receiving the spherical head 420 of the fixation screw 42. The locking rod 30 is received in the U-shaped slot 12 of the fastening seat 10 such that the locking rod 30 is rested on the press ring 422, and that the spherical head 420 is pressed and confined by the press ring 422, thereby enabling the locking rod 30 and the press ring 422 to have a linear contact instead of the point contact of the prior art devices.

As shown in FIG. 9, the device of the first preferred embodiment of the present invention is used in conjunction with a connection apparatus 50. The fastening seat 10 is provided with a deviation hole 19 for receiving an assembly ring 52 of the connection apparatus 50, which is used to assemble the press ring 422 and the spherical head 420. In order to fixedly connect the assembly ring 52 to the fastening seat 10, threads may be formed on the inner wall of the deviation hole 19 and on the outside surface of the assembly ring 52. Alternatively, threaded through holes perpendicular to the deviation hole 19 may be provided on the fastening seat 10 and fastening bolts engageable with the threaded through holes may be provided to fixedly connect the assembly ring 52 to the fastening seat 10.

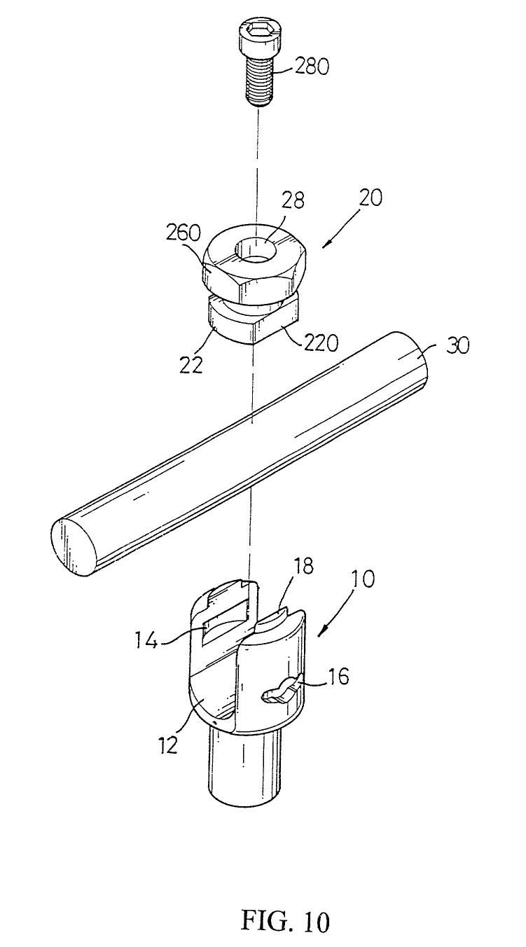

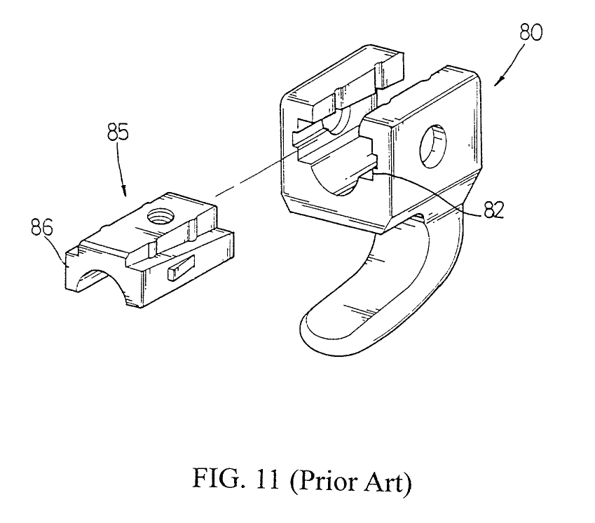

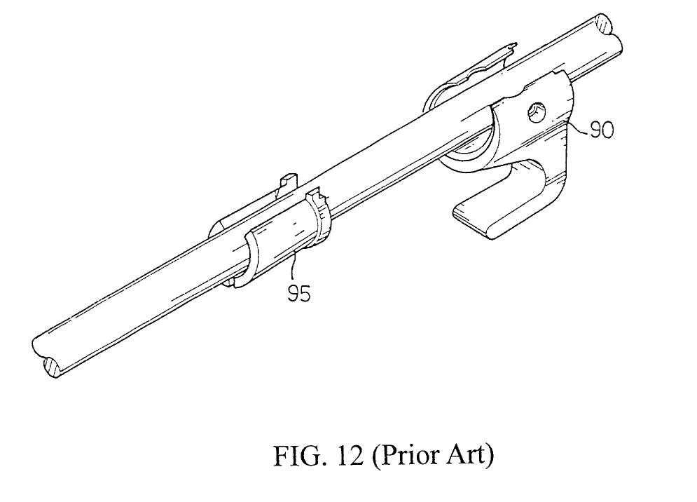

As shown in FIG. 10, a device of the third preferred embodiment of the present invention is basically similar in construction to that of the first preferred embodiment, except that the former comprises a fastening block 20 which is provided with a threaded hole 28. A fastening screw 280 is engaged with the threaded hole 28, thereby resulting in an extra pressure to exert on the locking rod 30.

The devices of the present invention are applicable to not only the locking rod but also the locking screw.

The embodiments of the present invention described above are to be regarded in all respects as being illustrative and nonrestrictive. Accordingly, the present invention may be embodied in other specific forms without deviating from the spirit thereof. The present invention is therefore to be limited only by the scopes of the following claims.

What is claimed is:

1. A rotary device for fastening a spine-locking rod or screw, said rotary device comprising:
a fastening seat provided with a U-shaped slot for receiving the spine-locking rod or screw, said U-shaped slot provided on two opposite inner walls with an inner retaining recess; and
a fastening block provided with a retaining flange which is provided in two opposite sides with a tangent surface, said fastening block further provided in a bottom with an arcuate press surface, and in a top with a rotating portion, wherein said fastening block is rotatably joined with said fastening seat such that said fastening block can be turned so as to enable said retaining flange to fit into said inner retaining recesses of said fastening seat, thereby causing said arcuate press surface of said fastening block to press against the spine-locking rod or screw which is received in said U-shaped slot of said fastening seat.

2. The rotary device as defined in claim 1, wherein said U-shaped slot of said fastening seat is provided on two opposite outer walls with an outer retaining recess to facilitate the holding of said fastening seat by a hand tool.

3. The rotary device as defined in claim 1, wherein said rotating portion is provided with a position-confining groove; wherein said U-shaped slot of said fastening seat is provided at a top of two side walls thereof with a position-confining protrusion, wherein said position-confining protrusion is fitted into said position-confining groove of said rotating portion of said fastening block at such time when said fastening block is mounted to said U-shaped slot of said fastening seat.

4. The rotary device as defined in claim 1, wherein said arcuate surface of said fastening block is provided with an assembly arcuate recess parallel to said tangent surface, and a fixation arcuate recess perpendicular to said assembly arcuate recess, with said assembly arcuate recess being greater in depth than said fixation arcuate recess.

5. The rotary device as defined in claim 4, wherein said arcuate press surface of said fastening block is further provided with an adjustment arcuate recess whereby said adjustment arcuate recess is located between said assembly arcuate recess and said fixation arcuate recess and is intermediate in depth between said assembly arcuate recess and said fixation arcuate recess.

6. The rotary device as defined in claim 1, wherein said fastening seat is provided with a fastening screw made integrally therewith.

7. The rotary device as defined in claim 1, wherein said fastening seat is provided with a clamp made integrally therewith.

8. The rotary device as defined in claim 1, wherein said fastening seat is provided with a receiving hole below the U-shaped slot, and is further provided with a connection apparatus comprises a spherical head and a press ring, wherein said spherical head and said press ring are adapted to be received in said receiving hole with said press ring resting on said spherical head.

9. The rotary device as defined in claim 8, wherein said receiving hole is a deviation hole, and said fastening seat is further provided with an assembly ring, wherein said an assembly ring which is adapted to mounted to said receiving hole so that said spherical head is clamped between said assembly ring and said press ring.

10. The rotary device as defined in claim 8, wherein said spherical head is provided with a fastening screw made integrally therewith, wherein said fastening screw is extruding from said receiving hole when said spherical head is received in said receiving hole.

11. The rotary device as defined in claim 9, wherein said spherical head is provided with a fastening screw made integrally therewith, wherein said fastening screw is extruding from said receiving hole when said spherical head is received in said receiving hole.

12. The rotary device as defined in claim 1, wherein said fastening block is provided with a threaded hole for threadably receiving a fastening screw for resulting an added pressure to exert on the spine-locking rod or screw.
